(19) United States
(12) Patent Application Publication
(10) Pub. No.: US 2014/0065210 A1
(43) Pub. Date: Mar. 6, 2014

(54) PROTEIN STABILIZED PROBIOTICS AND PERSONAL CARE PRODUCTS

(71) Applicants: David W. Koenig, Menasha, WI (US); Neil T. Scholl, Neenah, WI (US); Vasily A. Topolkaraev, Appleton, WI (US); Amy L. Vanden Heuvel, Hortonville, WI (US)

(72) Inventors: David W. Koenig, Menasha, WI (US); Neil T. Scholl, Neenah, WI (US); Vasily A. Topolkaraev, Appleton, WI (US); Amy L. Vanden Heuvel, Hortonville, WI (US)

(21) Appl. No.: 13/598,383

(22) Filed: Aug. 29, 2012

(51) Int. Cl. A61K 35/66; A61K 9/00; A61P 15/00; A61K 9/20; A61P 31/00; A61P 13/00; A61K 35/74; A61K 9/48

(52) U.S. Cl. 424/451; 424/93.1; 424/93.45; 424/93.51; 424/93.4; 424/93.48; 424/93.42; 424/93.44; 424/93.46; 424/484; 424/464

(57) ABSTRACT

The present invention relates to a personal care product comprising a protein matrix wherein said protein matrix comprises at least one protein, at least one probiotic and a carrier fluid.

The present invention also relates to the personal care product being a feminine care product particularly aimed at female urogenital health to treat or prevent vaginal infections. Oral and sinus infections, however, may also be benefited by the composition.

### FIELD OF THE INVENTION

The present invention relates to the stabilization of probiotics and their use in personal care products.

### BACKGROUND OF THE INVENTION

Probiotics and prebiotics have received growing attention and research over the years for the purpose of alleviating or improving certain health symptoms. The United Nations Food and Agriculture Organization (FAO) and World Health Organization (WHO) define probiotics as “living microorganisms, which, when administered in adequate amounts, confer a health benefit to the host” (2001). Prebiotics refer to nutrients which promote the growth of certain microorganisms, typically for the purpose of benefiting host health. Prebiotics and probiotics essentially work to enhance the growth of beneficial bacteria in the body, which thereby inhibit, neutralize, or out-compete pathogenic bacteria.

Homeostasis is the ability to control the environment of the body cavity or skin such that a climax micro-ecology is maintained. Infection is largely a function of the inability to maintain homeostasis. Without knowing what components are required to maintain homeostasis, it becomes very difficult to effectively fight infection and maintain health. Therefore, the use of probiotics and prebiotics to restore or maintain homeostasis to fight infection is important particularly in the development of applications that can provide certain treatments in this area.

Therefore, the present invention seeks to provide advanced technology solutions for the application of prebiotics and probiotics in personal care products. Additionally, the present invention seeks to provide such application while overcoming the storage and shelf life issues to maintain the viability of the probiotics.

### SUMMARY OF THE INVENTION

The present invention relates to a personal care product comprising a protein matrix wherein said protein matrix comprises at least one protein, at least one probiotic and a carrier fluid.

The present invention also relates to the personal care product being a feminine care product particularly aimed at female urogenital health to treat or prevent vaginal infections.

### DETAILED DESCRIPTION OF THE INVENTION

While the specification concludes with the claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore; do not include solvents or by-products that may be included in commercially available materials, unless otherwise specified. The term “weight percent” may be denoted as “wt. %” herein. Except where specific examples of actual measured values are presented, numerical values referred to herein should be considered to be qualified by the word “about”.

As used herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

As used herein, “homeostasis” is the ability to control the environment of the body cavity or skin such that a climax micro-ecology is maintained.

“Host” as used herein refers to a mammal, particularly a human who may benefit from the growth of microorganisms and their nutrients.

“Prebiotics”, as used herein, refers to nutrients which promote the growth of certain microorganisms, typically for the purpose of benefiting host health.

As used herein, “probiotics” are living microorganisms, which, when administered in adequate amounts, confer a health benefit to the host.

The present invention relates to a personal care product comprising a protein matrix wherein said protein matrix comprises at least one protein, at least one probiotic and a carrier fluid. The protein matrix allows for a low temperature melt process that provides an efficient and effective means of producing stabilized probiotics. Probiotics need to be stabilized within personal care products in order that they may survive during the transfer and storage of such products. Additionally, they must be able to sustain a particular shelf life that often accompanies consumer goods.

Low temperature extrusion methods are utilized to incorporate probiotics into a protein matrix. The probiotics can then be recovered from the extruded blends of the protein matrix.

### Protein Matrix

The protein matrix helps to protect the probiotic by maintaining an external environment such that Aw, osmotic pressure, oxidative stress or other factors that may damage or destroy the probiotic are kept away. In addition to protecting the probiotic, the protein matrix, also keeps within, other materials such as probiotic aids, carrier fluids, and any other components suitable as additives within the composition. Upon appropriate conditions, the probiotic will be released into the environment in an effort to work against any bacteria present, maintain a healthy environment within the host, or both.

Proteins suitable as the matrix of the present invention include vegetable proteins, dairy proteins, animal proteins, as well as concentrates or isolates thereof. The protein source may be, for instance, milk (e.g., casein or caeseinates), whey, corn (e.g., zein), wheat (e.g., wheat gluten), soy, or other vegetable or animal sources. Plant proteins are particularly suitable for use in the present invention, such as zein, corn gluten, wheat gluten, whey protein, soy protein, etc. Any form of protein may be used, such as isolates, concentrates and flour. For example, soy proteins may be in the form of an isolate containing from about 75 wt. % to about 98 wt. % protein, a concentrate containing from about 50 wt. % to about 75 wt. % protein, or flour containing from about 30 wt. % to about 50 wt. % protein. In certain embodiments, it is desirable to use a protein that is relatively pure, such as those having a protein content of about 75 wt. % or more, and in some cases, about 85 wt. % or more. Gluten proteins, for instance, may be purified by washing away any associated starch to leave a composite of gliadin and glutenin proteins. In one particular embodiment, a vital wheat gluten is employed. Such vital wheat gluten is commercially available as a creamy-tan powder produced from wheat flour by drying freshly washed gluten. For instance, vital wheat gluten can be obtained from Archer Daniels Midland (“ADM”) of Decatur, Ill. under the designations WhetPro® 75 or 80. Similarly, purified soy protein isolates may be prepared by alkaline extraction of a defatted meal and acid precipitation, a technique well-known and used routinely in the art. Such purified soy proteins are commercially available from ADM under the designation PRO-FAM®, which typically have a protein content of 90 wt. % or more. Other purified soy protein products are also available from DuPont of Louisville, Ky. under the designation PRO-COTE® and from Central Soya under the designation Promie R®.

If desired, the protein may also be modified using techniques known in the art to improve its ability to disperse in an aqueous solution. Suitable modification techniques may include pH modification, denaturation, hydrolysis, acylation, reduction, oxidation, etc. Just as an example, gluten may sometimes absorb water until it begins to repel excess water. This results in gluten molecules that are associated closely together such that they resist dispersion in aqueous solutions. To counteract this tendency, the protein may be treated with a pH modifier to increase its solubility in aqueous environments. Typically, the pH modifier is a basic reagent that can raise the pH of the protein, thereby causing it to become more soluble in aqueous solutions. Monovalent cation-containing basic reagents (hereafter “monovalent basic reagents”) are particularly suitable for use in the present invention. Examples of such monovalent basic reagents include, for instance, alkali metal hydroxides (e.g., sodium hydroxide, ammonium hydroxide, etc.), ammonia, etc. Of course, multivalent reagents, such as alkaline metal hydroxides (e.g., calcium hydroxide) and alkaline metal oxides (e.g., calcium oxide), may also be employed if desired. When employed, the pH modifier may be present in an amount such that the pH of the protein is from about 7 to about 14, and in some embodiments, from about 8 to about 12.

Hydrolysis of the protein material may also improve water solubility, and can be affected by treating the protein with a hydrolytic enzyme. Many enzymes are known in the art which hydrolyze protein materials, including, but not limited to, proteases, pectinases, lactases, and chymotrypsin. Enzyme hydrolysis is affected by adding a sufficient amount of enzyme to an aqueous dispersion of protein material, typically from about 0.1% to about 10% enzyme by weight of the protein material, and treating the enzyme and protein dispersion. After sufficient hydrolysis has occurred the enzyme may be deactivated by heating, and the protein material may be precipitated from the solution by adjusting the pH of the solution to about the isoelectric point of the protein material.

The matrix of the present invention may use proteins in an amount of from about 30 wt. % to about 95 wt. %, in some embodiments from about 40 wt. % to about 90 wt. %, and in some embodiments, from about 50 wt. % to about 80 wt. %.

Probiotics

Probiotics are added to the composition to confer a health benefit to the host.

Suitable probiotics of the present invention include, but are not limited to, Lactobacillus, Saccharomyces, Bifidobacterium, Pediococcus, Leuconostoc, Micrococcus, Escherichia, Staphylococcus, Streptococcus, Candida, and Bacillus, and combinations thereof. The amount could be as little as one viable microbe (probiotic) delivered to the host anatomy of interest. From about 1×10⁵ colony-forming units to about 1×10⁹ colony-forming units can be delivered to the host site.

The probiotics of the present invention are preferably freeze-dried or otherwise in a state of rest within the composition. Other non-freeze dried bacteria, however, may be used within the present invention such as Bacilus spores or stabilized vegetative bacteria. Stabilizing vegetative bacteria, as used, herein refers to bacteria coated with glycerol or hydrophobic polymer coatings.

The probiotics of the present invention begin to move from a rested state and begin to grow once they are exposed to an approximate water activity (Aw) greater than about 75% and will move to target the host bacteria. The secretion of toxins within the bacteria area also helps the probiotics to populate. The composition is particularly suitable for the vaginal region, the nasal mucosa or the oral area of the host. When the probiotic of the composition reaches the particular host problem area, the resident flora is able to recolonize to a diversity and richness that is equal to the previous healthy state in order to allow healing. The probiotic is the catalyst for this happening by shifting the ecology to allow for regrowth of the host bacteria.

Probiotic Aids

Additional components may be added to the composition of the present invention to help maintain the viability or prevent the destruction of the probiotic within the protein matrix. Probiotic aids of the present invention may be selected from moisture scavengers, osmotic stabilizers, antioxidants, prebiotics, phase change materials and combinations thereof.

Moisture Scavengers

Moisture scavengers can be added to the composition of the present invention to decrease and maintain a low water activity level, preferably below Aw of 70% that helps to maintain the viability of the microorganism until use. Moisture scavengers can be classified as any compound that binds, holds, or reacts with water that results in a lower water activity level within the composition of the present invention. Suitable moisture scavengers of the present invention include, but are not limited to, oxazolidine, calcium oxide, zeolite, calcium sulfate, calcium carbonate, lithium, sodium, potassium, caesium, and combinations thereof. The moisture scavengers of the present invention may be added in an amount from about 0.01% to about 1%, by weight of the composition.

Osmotic Stabilizers

Osmotic stabilizers can be added to the composition to help stabilize the probiotics by preventing water loss to the environment and/or an influx of water into the membrane which could damage or kill the probiotic. The Osmotic stabilizers will help to stabilize the biological membranes surrounding the probiotic against reduced water or increased salts. Although the probiotics are in a state of rest during shipping, storage, and the like, any water or salt intrusion upon the membrane would cause damage to the probiotic which may reduce the efficacy of the composition. Suitable osmotic stabilizers of the present invention include, but are not limited to glycerol, ethylene glycol, sucrose, NaCl, chlorpromazine, proline, thiopental, tetracine phosphatidylcholines (PCs), phosphatidylethanolamines (PEs), phosphatidylglycerols, peptones, tryptones, meat extract, sorbitol, glutamate, mannitol, exopolysaccharides (EPS), amino acids, low molecular weight polymers, mannose, galactose, adonitol, glutamate, raffinose, lactose, fructose, trehalose, inositol, dehydrins, and combinations thereof. The osmotic stabilizers of the present invention may be added in an amount from about 0.01% to about 1%, by weight of the composition.

Antioxidants

Antioxidants may protect the probiotic within the present invention by inhibiting the oxidation of other molecules or reducing the effects of oxidants. Like water or salt intrusion, oxidative damage could also occur if an oxidant is able to harm or penetrate the membrane and cause damage to the probiotic. Antioxidants can be added to the composition to help prevent oxidative damage to the probiotics. Suitable antioxidants of the present invention include, but are not limited to sodium ascorbate, tocopherols, thiols, polyphenols, ascorbic acid, propyl-gallate, and combinations thereof. Antioxidants may be added to the composition in an amount from about 0% to about 10%, by weight to the weight of composition, or from about 0.5% to about 5% by weight of the composition, or from about 1% to about 3% by weight of the composition.

Prebiotics

Prebiotics can be added to the composition to benefit the host by stimulating growth of the probiotics. Prebiotics are non-digestible food ingredients. Suitable prebiotics of the present invention include, but are not limited, to inulin molecules of all chain lengths, xylitol, lactulose, tagatose, gluco-oligosaccharides, fructooligosaccharides, xylooligosaccharides, trans-galacto-oligosaccharides, galacto-oligosaccharides, and combinations thereof. The following botanicals are also suitable prebiotic sources chicory root, Jerusalem artichoke, dandelion greens, garlic, leek, onion, asparagus, wheat bran, whole wheat flour, banana, and combinations thereof. Prebiotics of the present invention may be added into the composition in an amount from about 0.01% to about 10% by weight of the composition.

Phase Change Materials

Because the viability of the probiotics within the composition can be affected by increased temperatures, such as during shipping and storage, phase change materials can be incorporated into the composition to help maintain a desired temperature and help maintain a viable probiotic. Phase change materials help to absorb the heat that can be stored or released during shipping and storage and helps protect the probiotic from the adverse effects of the environment. Phase change materials also take advantage of latent heat that can be stored or released from a material over a narrow temperature range. Thus, they are useful in the present invention because they are able to absorb energy that helps maintain a temperature environment suitable for probiotic survival. Suitable phase change materials of the present invention include, but are not limited to, hydrated inorganic salts such as hydrated sodium sulfate, Manganese(II) nitrate hexahydrate, linear long chain hydrocarbons, polyethylene glycol (paraffin waxes), and combinations thereof. Phase change materials may be added to the composition in an amount from about 0% to about 30% by weight of composition, or from about 5% to about 25% weight of composition, or from about 10% to about 20% by weight of composition.

Carrier Fluid (Plasticizer)

A carrier fluid or plasticizer may be used within the matrix of the present invention to help render the protein more flowable under melt processing conditions and able incorporate the “microorganism” into the composition. Suitable carrier fluids may include, for instance, polyhydric alcohols, such as sugars (e.g., glucose, sucrose, fructose, raffinose, maltodextrose, galactose, xylose, maltose, lactose, mannose, and erythrose), sugar alcohols (e.g., erythritol, xylitol, malitol, mannitol, and sorbitol), polyols (e.g., ethylene glycol, glycerol, propylene glycol, dipropylene glycol, butylene glycol, and hexane triol), and combinations thereof. Also suitable are hydrogen bond forming organic compounds which do not have hydroxyl group, including urea and urea derivatives; anhydrides of sugar alcohols such as sorbitan; animal proteins such as gelatin; vegetable proteins such as sunflower protein, soybean proteins, cotton seed proteins; and mixtures thereof. Other suitable carrier fluids may include phthalate esters, dimethyl and diethylsuccinate and related esters, glycerol triacetate, glycerol mono and diacetates, glycerol mono, di, and tripropionates, butanoates, stearates, lactic acid esters, citric acid esters, adipic acid esters, stearic acid esters, oleic acid esters, and other acid esters. Aliphatic carboxylic acids may also be used, such as lactic acid, maleic acid, acrylic acid, copolymers of ethylene and acrylic acid, polyethylene grafted with maleic acid, polybutadiene-co-acrylic acid, polybutadiene-co-maleic acid, polypropylene-co-acrylic acid, polypropylene-co-maleic acid, and other hydrocarbon based acids. A low molecular weight carrier fluid is preferred, such as less than about 20,000 g/mol, preferably less than about 5,000 g/mol and more preferably less than about 1,000 g/mol.

The degree of water solubility of the carrier fluid can vary. Carrier fluids can have infinite solubility in water to not being water soluble at all. The more hydrophobic the carrier fluid can help maintain a lower water of activity level compared to carrier fluids that have hydrophilic properties.

The amount of the carrier fluids employed depends in part on the nature of the selected protein, but is typically from about 1 wt. % to about 50 wt. %, in some embodiments from about 5 wt. % to about 30 wt. %, and in some embodiments, from about 10 wt. % to about 20 wt. %.

### Melt Processing Technique

As indicated above, the composition of the present invention is formed by processing the components together in a melt blending device (e.g., extruder). The mechanical shear and heat provided by the device allows the components to be blended together in a highly efficient manner without the use of a solvent. Batch and/or continuous melt blending techniques may be employed in the present invention. For example, a mixer/kneader, Banbury mixer, Farrel continuous mixer, single-screw extruder, twin-screw extruder, roll mill, etc., may be utilized. One particularly suitable melt-blending device is a co-rotating, twin-screw extruder (e.g., USALAB twin-screw extruder available from Thermo Electron Corporation of Stone, England or an extruder available from Werner-Pfleiderer from Coperion Ramsey, N.J.). The raw materials (e.g., microorganism, protein, carrier fluid, etc.) may be supplied to the melt blending device separately and/or as a blend. For example, the protein and/or microorganism may be initially fed to a feeding port of the twin-screw extruder. Thereafter, a carrier fluid may be injected into the extruder downstream from the microorganism and protein. Alternatively, the components may be simultaneously fed to the feed throat of the extruder or separately at a different point along its length.

The materials are dispersively blended under low shear/pressure and at a low temperature to minimize protein dissociation associated with aggregation and maintain microorganism viability. Nevertheless, the temperature is still typically slightly at or above the softening point of the protein. For example, melt blending typically occurs at a temperature of from about 20° C. to about 60° C., in some embodiments, from about 25° C. to about 50° C., and in some embodiments, from about 30° C. to about 40° C. Likewise, the apparent shear rate during melt blending may range from about 100 seconds⁻¹ to about 5,000 seconds⁻¹, in some embodiments from about 200 seconds⁻¹ to about 2,000 seconds⁻¹, and in some embodiments, from about 400 seconds⁻¹ to about 1,200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows. The apparent melt viscosity of the resulting composition may be relatively low, such as from about 1 to about 100 Pascal seconds (Pa·s), in some embodiments from about 5 to about 60 Pa·s, and in some embodiments, from about 20 to about 50 Pa·s, as determined at a temperature of 160° C. and a shear rate of 1000 sec⁻¹. The melt flow index (190° C., 2.16 kg) of the composition may also range from about 0.05 to about 50 grams per 10 minutes, in some embodiments from about 0.1 to about 15 grams per 10 minutes, and in some embodiments, from about 0.5 to about 5 grams per 10 minutes.

### Forms for Applying to Article

Once formed, the composition of the present invention may be used in a variety of articles selected from a lotion, cream, jelly, powder, liniment, ointment, salve, oil, foam, gel, film, wash, coating, liquid, capsule, tablet, concentrate, fibers and the like. Most notably, the present invention may be a feminine care product particularly aimed at female urogenital health to treat or prevent vaginal infections. Both oral and nasal infections are also benefited by the composition of the present invention.

Besides being formed into a film, the composition of the present invention may also be formed into particles and applied to other types of articles. Powderization may be accomplished using any of a variety of known techniques.

Regardless of its particular form, the particles may be applied to a wide variety of different articles to deliver the microorganism.

The composition can also be formed into an article such as an implant to deliver the microorganism.

Methods for Probiotic Extraction

In order to determine the colony forming units per gram of composition (CFU/g) contained within the extruded material, a measured amount of the extruded material may be placed in a centrifuge tube with a buffered saline solution. Various codes can then be plated onto agar along with a negative control for comparison. Serial dilutions are then performed from the extraction tubes and the colony forming units are counted. The average CFU can be multiplied by the dilution factor to determine the average CFU/mL. This number can then be used to calculate the CFU/g of material by multiplying by the volume of the buffered saline solution added to the extruded material. This is further explained by way of the examples and test methods shown below.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

Probiotic Extraction Test Methods

To determine the colony forming units per gram (CFU/g) contained within extruded material, 1 g of extruded material was weighed out in triplicate per code and placed in sterile 15-mL conical centrifuge tubes. 10 ml of phosphate buffered saline (PBS) was added to each tube. 100 μl of Tween 80 was also added to each tube. The tubes were vortexed for 30 sec, then sonicated 5 min with 1 minute rest intervals after 1 minute of sonication. Codes were diluted in PBS and plated onto appropriate media. Lactobacillus codes and capsules were plated onto Lactobacilli MRS agar. Saccharomyces codes and capsules were plated onto Sabouraud dextrose (Sab dex) agar. A negative control code was extruded without probiotics and was plated on MRS agar, Sab dex agar, and Tryptic soy agar plates.

Serial dilutions were performed from the extraction tubes. PBS was the diluent used. CFU/g were determined by counting the colony forming units (CFU) on the plates. The average CFU was calculated based on the CFU counted from duplicate plate dilutions. The average CFU was multiplied by the dilution factor to determine the average CFU/mL. The calculated CFU/mL was multiplied by 10 to calculate the CFU/g. (CFU/mL was multiplied by 10 to account for the 10 mL of PBS added to 1 gram of extruded material.)

### Example 1

Blending of wheat gluten (WhetPro® 75 from Archer Daniels Midland Company), Glycerol (Emery 917 Glycerine 99.7% USP from Cognis Oleochemicals), and Lactobacillus acidophilus (Florejen®) was conducted by utilizing Thermo Prism Usalab 16 lab scale co-rotating twin screw extruder that allows for temperature control of 9 different zones. Before blending, the Thermo Prism extruder was heated to 220° C. for 30 minutes to sterilize and allowed to cool with openings covered with aluminum foil to prevent contamination. The hopper and funnel were disinfected with 70 wt. % Isopropanol and covered with aluminum foil until use. Extrusion process was absent of die to allow ease of material exit, and zones 1 through 5 were not utilized. Dry blend of wheat gluten contained 1.00E+07 CFU/g of Lactobacillus acidophilus was feed at zone 6 via drop feeder at approximately 0.5 lbs./hr. and glycerol was fed at zone 7 via liquid injection pump at a feed rate of approximately 0.2 lbs./hr. The screw configuration was composed of conveying elements at zones 6 and 7, kneading blocks at 8 and 9, and conveying elements at Zone 10. Heat source for the barrels was turned off to keep temperatures as low as possible. Processing temperatures reached 38° C. To determine the viability of probiotics, extruded material was accelerated aged at 50° C. Results of Lactobacillus CFU/g of extruded material before and after aging are set forth in Table 1.

### Example 2

Same as Example 1, except dry blend of wheat gluten contained 1.00E+08 CFU/g of Lactobacillus acidophilus. Processing temperatures reached 42° C.

### Example 3

Same as Example 1, except dry blend of wheat gluten contained 1.00E+09 CFU/g of Lactobacillus acidophilus. Process temperatures reached 43° C.

### Example 4

Same as Example 3, except soy protein flour from Archer Daniels Midland Company was utilized in replace of wheat gluten. Process temperature reached 43° C.

### Example 5

Same as Example 3, except poly(ethylene glycol) with an average molecular weight of 200 (Sigma Aldrich) was utilized in replace of glycerol. Process temperature reached 40° C.

### Example 6

Same as Example 3, except L(+) Lactic acid (Purac® FCC 88) was utilized in replace of glycerol. Process temperature reached 40° C.

### Example 7

Same as Example 3, except triethanolamine (Aldrich) was utilized in replace of glycerol. Process temperature reached 30° C.

### Example 8

Same as Example 3, except tributyl citrate (Aldrich) was utilized in replace of glycerol. Process temperature set at 50° C.

### Example 9

Same as Example 3, except tributyl citrate (Aldrich) was utilized in replace of glycerol. Process temperature reached 30° C.

### Example 10

Same as Example 3, except triethylcitrate (Aldrich) was utilized in replace of glycerol. Process temperature set at 50° C.

### Example 11

Same as Example 10.

### Example 12

Same as Example 10, except dry blend of wheat gluten contained 1.00E+08 CFU/g of Saccharomyces boulardii in replace of lactobacillus acidophilus. Process temperature set at 50° C.

### Example 13

Same as Example 10, except no probiotic microorganisms were added to wheat gluten. Process temperature set at 50° C.

TABLE 1

| | | | Average | Average | |
|---|---|---|---|---|---|
| | | Probiotic | Probiotic | Probiotic | Average |
| | Day 0 | Recovery | Recovery | Recovery | Probiotic |
| | Average | after 7 | at 50° C. | at 50° C. | Recovery at |
| | Probiotics | days at | for 14 | for 21 | 50° C. for 29 |
| | Recovered | 50° C. (log | days (log | days (log | days (log |
| EXAMPLE | (log CFU/g) | CFU/g) | CFU/g) | CFU/g) | CFU/g) |
| 1 | 2.8 | <1.0* | — | NT | NT |
| 2 | 6.2 | <1.0* | — | NT | NT |
| 3 | 6.1 | <1.0* | NT | NT | NT |
| 4 | 4.5 | <1.0* | NT | NT | NT |
| 5 | <2.0* | NT | NT | NT | NT |
| 6 | <2.0* | NT | NT | NT | NT |
| 7 | 4.5 | <2.0* | NT | NT | NT |
| 8 | 7.3 | 5.6 | NT | NT | NT |
| 9 | 7.3 | 4.9 | NT | NT | NT |
| 10 | 7.0 | 4.6 | NT | NT | NT |
| 11 triethylcitrate-lac | 8.1 | 3.1 | 3.0 | 3.3 | 4.1 |
| 12 triethyl - bou | 8.8 | 7.6 | 6.2 | 5.6 | 6.9 |
| 13 triethyl - control | <2.0* | NT | NT | NT | NT |

*Below detection limits
NT = not tested

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A personal care product comprising a protein matrix wherein said protein matrix comprises at least one protein, at least one probiotic and a carrier fluid.

2. The personal care product of claim 1 wherein said probiotic is selected from Lactobacillus, Saccharomyces, Bifidobacterium, Pediococcus, Leuconostoc, Micrococcus, Escherichia, Staphylococcus, Streptococcus, Candida, and Bacillus, and combinations thereof.

3. The personal care product of claim 1 wherein said protein matrix consists of protein selected from vegetable proteins, dairy proteins, animal proteins, isolates thereof, and combinations thereof.

4. The personal care product of claim 3 wherein said protein is present in an amount from about 30% to about 95%, by weight of the composition.

5. The personal care product of claim 4 further comprising a pH modifier such that the pH of the protein is from about 7 to about 14.

6. (canceled)

7. The personal care product of claim 1 wherein the probiotic is present in an amount of from about 1×10⁵ CFU/g to about 1×10⁸ CFU/g, by weight of the protein matrix.

8. The personal care product of claim 1 further comprising probiotic aids selected from moisture scavengers, osmotic stabilizers, antioxidants, prebiotics, phase change materials and combinations thereof.

9. The personal care product of claim 1 further comprising a carrier fluid having a molecular weight of less than about 20,000 g/mol selected from the group consisting of polyhydric alcohols, sugar alcohols, polyols, and combinations thereof.

10. The personal care product of claim 1 formed within an article selected from a lotion, cream, jelly, powder, liniment, ointment, salve, oil, foam, gel, film, wash, coating, liquid, capsule, tablet and fibers.

11. The personal care product of claim 10 wherein the personal care product is a feminine care product designed to treat or prevent vaginal infections.

12. (canceled)

13. (canceled)
